(19) United States
(12) Patent Application Publication
(10) Pub. No.: US 2022/0000339 A1
(43) Pub. Date: Jan. 6, 2022

(54) DIAGNOSTIC CERVICAL SCANNING AND TREATMENT DEVICE

(71) Applicant: Maine Medical Center, Portland, ME (US)

(72) Inventor: Obieze Nwanna-Nzewunwa, Portland, ME (US)

(21) Appl. No.: 17/367,892

(22) Filed: Jul. 6, 2021

Related U.S. Application Data
(60) Provisional application No. 63/048,315, filed on Jul. 6, 2020.

(51) Int. Cl. A61B 1/00; A61B 1/05; A61B 1/045; A61B 18/02; G16H 50/70; G16H 50/20

(52) U.S. Cl. CPC A61B 1/00009; A61B 1/05; A61B 1/00016; A61B 2018/00559; A61B 18/02; G16H 50/70; G16H 50/20; A61B 1/045

(57) ABSTRACT

A device to screen for and treat cervical precancerous lesions combines features in an examination and treatment device, including an examination portal defined by an elongated body adapted for visual observation of an examination area, and an imaging apparatus disposed in an interior of the examination portal for capturing images of the examination area. An automated analysis with higher diagnostic accuracy may also be performed. A treatment probe is adapted for access to the examination area, such that the treatment probe is responsive to operator control for remedial medical contact with the examination area. Premalignant and some malignant lesions can often be treated by surface remedies such as electrocautery, cryotherapy, laser therapy, chemical ablation, or excision. The treatment probe is operable from the operator position concurrently with rendering of the captured images, combining the diagnosis and treatment procedures in the same examination, visit or transaction.

### RELATED APPLICATIONS

This patent application claims the benefit under 35 U.S.C. § 119(e) of U.S. Provisional Patent App. No. 63/048,315, filed Jul. 6, 2020 entitled “DIAGNOSTIC CERVICAL SCANNING AND TREATMENT DEVICE” incorporated herein by reference in entirety.

### BACKGROUND

Healthcare access in underserved and developing regions may often be limited and erratic. Female reproductive health services like routine gynecological examinations, screening procedures and tests are often lacking. Accordingly, gynecological diseases like cervical cancer and precancerous lesions that would otherwise be routinely detected and treated via early intervention in developed nations can go undetected. Irregularity of visits by competent medical staff, particularly doctors, can impede follow-up treatment even if an anomaly is detected. In can be problematic to track patients for return visits. Hence, the World Health Organization recommends the use of a “test and treat” approach using visual inspection with acetic acid or Lugol's iodine for diagnosis.

There is a need for a cervical cancer screening test that is affordable, accessible, quick, and easily linked to treatment; one that does not require specialists. Such an approach would mitigate loss to follow up.

### SUMMARY

A handheld point of care device to screen for and treat cervical precancerous and cancerous lesions which combines features in an examination and treatment device, including an examination portal defined by an elongated body adapted for visual observation of an examination area, and an imaging apparatus disposed in the interior of the examination portal for capturing images of the examination area, which is displayed on a screen. An applicator probe, in the form of a spray nozzle connected to a spray cannister containing the diagnostic reagent (e.g. acetic acid or Lugol's iodine) is activated by a trigger mechanism and directed jet of the reagent is applies to the target surface. This cause the target lesions to exhibit a diagnostic color change that can be identified by the human eye or enhanced by a machine learning diagnostic algorithm adapted to this device and cervical cancer and precancerous lesions. A treatment probe is adapted for access to the examination area, such that the treatment probe is responsive to operator control for remedial medical contact with the examination area. Precancerous lesions can often be treated by surface remedies such as electrocautery, laser therapy, chemical ablation, cryotherapy and sharp excision with a blade. The treatment probe is operable from the operator position concurrently with rendering of the captured images, combining the diagnosis and treatment procedures in the same visit or transaction. In contrast to conventional approaches, visual diagnosis may be augmented by a diagnostic image recognition, software application and/or machine learning algorithm. This provides that diagnosed patients do not need to subsequently seek a treatment procedure at a later time when logistic and other complexities may compromise treatment. In other words, in contexts where a required second treatment visit may never occur due to the poor access, cultural and social stigma, limited medical resources and other patients-related factors. Concurrent diagnoses and treatment alleviates this problem.

Cervical cancer is the 4th leading cancer in females worldwide. Over 80% of cases occur in rural settings with poor access to healthcare. This is likely attributable to unavailability of early screening and treatment at a precancerous stage. The disclosed device offers a point of care test and treat option for remote and underserved environments. After positioning the device adjacent to the cervical area, rendering of the captured images and activation of the treatment probe occur during a single examination. Visual inspection aided by the imaging device facilitates diagnoses based on appearance. Additionally, the machine learning diagnostic algorithm can be applied to the captured image to facilitate a diagnosis with improved accuracy relative to visual inspection by the human eye. The operator disposes the elongated body adjacent the examination area for access via the examination portal, which has a tubular shape for providing a viewpoint for the imaging device and access for the treatment probe.

Upon usage, the imaging apparatus renders an image depicting a color of an examined tissue portion. Typically, the color can be attributed to a detected premalignant or malignant lesions in the tissue. Either visual inspection or image processing of shade, shape and appearance can determine a likely match with malignant tissue for a concise determination of needed treatment. If the tissue appears precancerous or malignant, typically denoted by a color change in the tissue triggered by the chemical reagent used. In the case of acetic acid, it elicits a white or greyish appearance in contrast to a healthy pink, the treatment probe is employed for treatment.

A diagnostic applicator, spray, swab or brush may be used to apply a diagnostic imaging substance, such as acetic acid or vinegar, which aids in coloring the examined tissue based on the presence of precancerous tissue or overt malignancy. If the diagnosis is positive, the remedial surgical/medical contact includes at least one of electrocautery, chemical ablation, cryotherapy, or surgical excision administered via the probe, but could also be application of medicinal treatments to the surface of the afflicted area. An electrocautery approach can be administered by a low voltage heating filament at a distal end of the treatment probe. On board batteries can provide sufficient power, providing diagnosis and treatment in a single exchange.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a side view of an examination and treatment device suited for cervical screening as defined herein;

FIG. 2 is an example of images gathered by the device of FIG. 1 employed in the screening and diagnostic determination;

FIG. 3A is a schematic view of the device of FIG. 1;

FIG. 3B is a front view of the elongated body of the device as in FIG. 3A; and

FIG. 4 is a flowchart of usage, diagnosis and treatment using the device of FIGS. 1-3B.

### DETAILED DESCRIPTION

Configurations below describe implementation of a portable, self-contained screening/diagnostic and treatment device for cervical cancer. Cervical cancer is a affects women who may often appear otherwise healthy, and mitigation through early detection and intervention is critical. The device is portable and well suited to rural and underdeveloped environments because it requires no external power, heavy machinery, or hospital resources and can therefore be easily transported and employed on site at remote locations. The disclosed device therefore provides a cervical cancer screening test that is inexpensive, accessible, quick, and provides diagnostics and treatment in the same transaction without a dedicated hospital setting. The disclosed configuration therefore provides a low-cost, hand-held point-of-care device to test and treat cervical pre-cancer and cancerous lesions in the same gynecological exam.

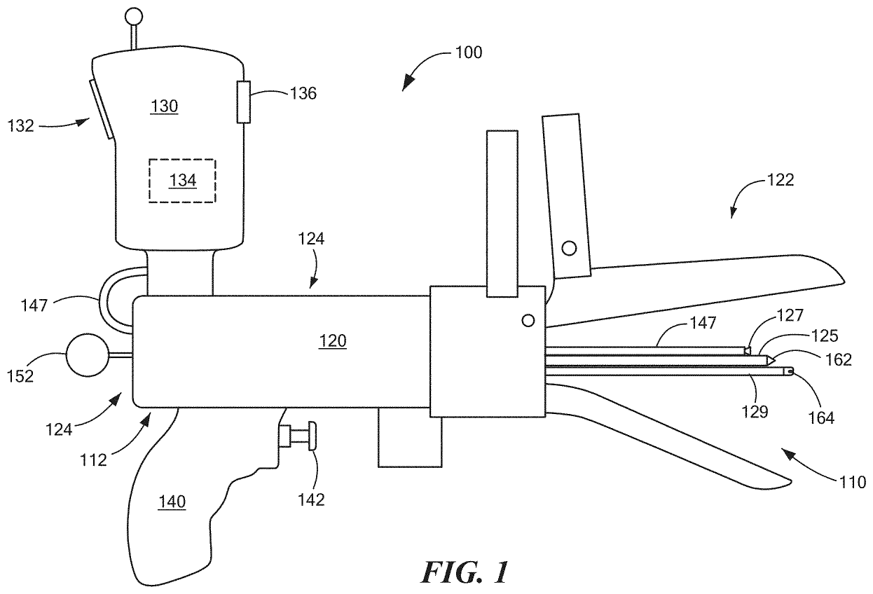

FIG. 1 is a side view of an examination and treatment device suited for cervical screening as defined herein. The cervical examination and treatment device 100 includes an examination portal 110 for examination site access defined by an elongated body 112 adapted for visual observation of an examination area. The examination portal is implemented in the form of an elongated tube 120 having a distal end 122 which fits into a detachable speculum with jaws 160-1 and 160-2 that open and close by the action of approximating or separating the levers 163 and 165. The jaws of the speculum 160-1 and 160-2 are inserted and used for displacement of soft tissues proximate to the examination area to facilitate exposure of the examination area and a proximate end 124 defining an operator position. A diagnostic applicator 125, imaging sensor, 127 and treatment probe 129 are concurrently disposed at the distal end 122 of the examination portal 110. Each of these three components performs distinct operations during the examination, diagnosis, and treatment transactions. The position of these three components is adjustable by the action of manipulating a controller/joystick 152 located at the rear end of the examination portal 120. The imaging sensor 127 is disposed in an interior of the examination portal for capturing images of the examination area, such that the imaging sensor is coupled to an imaging apparatus 130 including a screen 132 and optional image processing applications 134 which runs the machine learning diagnostic algorithmic analysis to provide a diagnosis on the captured images and connects to a database interface 136.

A diagnostic applicator 125 is also disposed in an interior of the examination portal for administering a reagent such as acetic acid to the examination area to facilitate imaging and diagnosis. Any suitable imaging reagent may be employed for causing a visual change in the examination area to indicate precancerous lesions, malignancies or other ailments. An imaging reagent reservoir 140 employs an actuator 142 for spraying or depositing the acetic acid or other imager from the diagnostic applicator 125 onto the examination area. Following deposition of the imaging agent, and based on the resulting image, the treatment probe 129 may be invoked to access the examination area, such that the treatment probe is responsive to an operator control for remedial surgical contact with the examination area. In response to a positive test or precancerous diagnosis resulting from the image inspection, the treatment probe is operable from the operator position concurrently with rendering of the captured images for cauterizing or ablating the examination area to mitigate and/or remove the identified tissue.

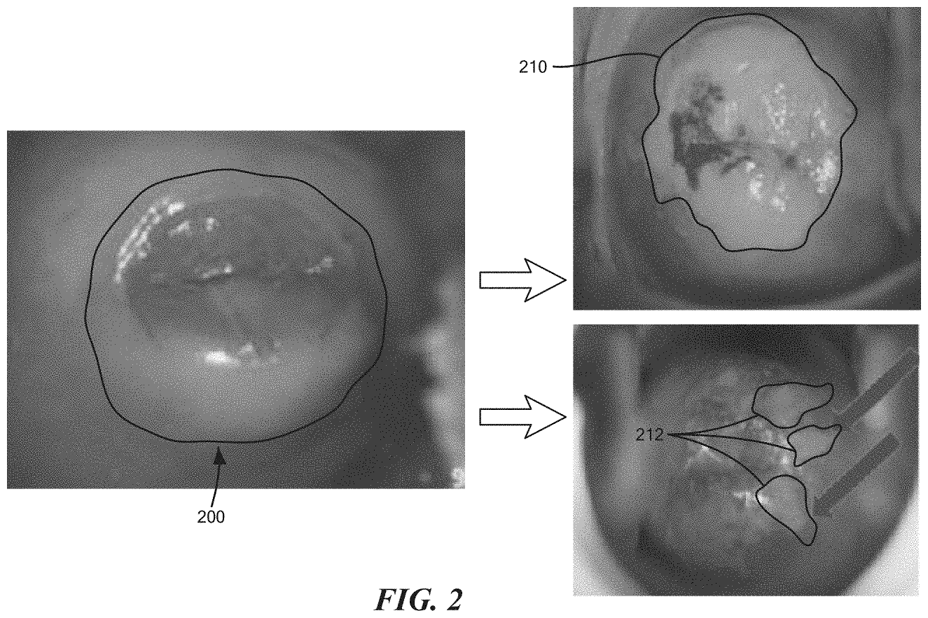

FIG. 2 is an example of images gathered by the device of FIG. 1 employed in the screening and diagnostic determination. Referring to FIGS. 1 and 2, upon patient examination, the imaging apparatus 130 renders an examination image on the screen 132 from the examination area. Upon application of the imaging agent onto an examination area 200, premalignant and malignant lesions are indicated by a whitening, lightening or pink color change in response to the imaging agent, shown by region 210 or patches/stripes 212. With the examination portal 110 still disposed in the examination area, the imaging sensor 127 couples to the imaging apparatus 130 via a signal cable 147 running through an interior of the elongated tube 120. The imaging apparatus 130, attached on top of the proximate end 124, is disposed for rendering an image depicting a shade of an examined tissue portion, such that the depicted shade or color is indicative of a detected malignancy of the examined tissue portion. The immediately visible image 132 on the imaging apparatus 130 allows visual examination, and may also be used for comparison images side by side on the display, or may perform image processing for matching with healthy and unhealthy images for automating the diagnosis.

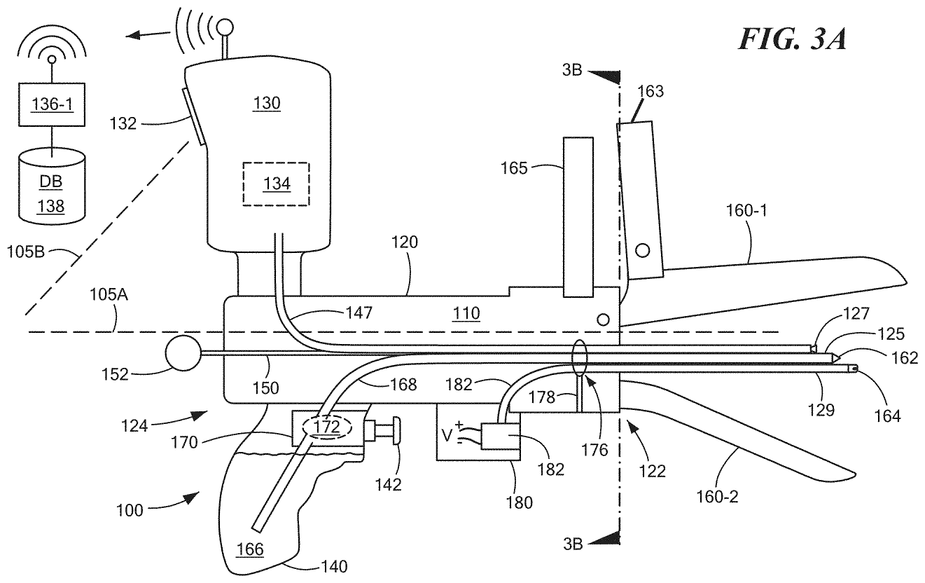

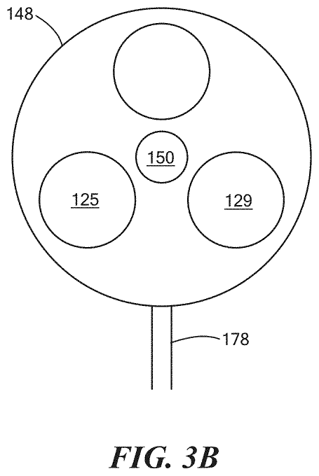

FIG. 3A is a schematic view of the device of FIG. 1, and FIG. 3B is a front view of the elongated body of the device as in FIG. 3A. Referring to FIGS. 1, 3A and 3B, the treatment device 100 is physically shaped for a cervical exam, having one or more wings 160-1 . . . 160-2 (160 generally) hingedly attached to the distal end 122 of the elongated tube 120 for temporarily displacing soft tissue around the examination area for providing unobstructed access to the examination area. The wings may be either fixed or hinged, and operate as a speculum to brace or dispose soft tissue through leverage from a handle 163 and fixed brace 165 for pivoting the wing 160-1 outward.

Once the wings 160 allow placement of the elongated tube 120, a line of sight 105A is established for invoking the diagnostic applicator 125, imaging sensor 127, and treatment probe 129. The diagnostic applicator 125, imaging sensor 127 and treatment probe 129 form a bundle 148 coupled to a control member 150 for operator manipulation. The control member 150 extends through the elongated body to the proximate end 124 and is responsive to operator control of the rigid bundle 148 via a handle 152.

The examination portal 110 defines a combined access for the diagnostic and treatment and also the line of site 105A for visual perspective. To facilitate, the elongated body 112 has a pivotal attachment 176 on a fulcrum 178 to the control member 150, such that the control member further includes a handle 152 at the proximate end of the elongated body, in which the distal end is responsive to manipulation from the handle via the pivotal attachment 176. The pivotal attachment 176 include a post extending from an interior side of the tube 120, an orifice in a transverse member running the diameter, or any suitable attachment to allow handle 152 driven manipulation of the control member 150 and tethered/attached instruments 125, 127, 129.

Collectively, these define a comingled treatment probe, including the diagnostic applicator 125, which has an imaging nozzle 162 at a distal tip adapted to dispense an imaging reagent onto the examination area. The imaging sensor 127 is configured for visual recordation of the examination area, and is more proximate than the tip of the imaging nozzle 162, otherwise the image might receive some of the spray from the nozzle 162 that would interfere with the image. The treatment probe 129 is for therapeutic contact with the examination area, and is more distal than the visual fiber 147 and imaging sensor 127 or the imaging nozzle 162 because treatment is effected by a tungsten filament 164 or similar electrocautery element or ablation (chemical, laser, blade, cryotherapy) technique contacting the tissue in the examination region 200.

Discussing the components of the control member 150 in more detail, an imager reservoir 140 is mounted on the device 100 for storing the imaging agent 166. Typically this is acetic acid, however any suitable imaging substance to result in a coloration or shading change to facilitate discrimination between portion of the examination that are positive in contrast to normal portions of the examination area could be employed. For example, a test involving Lugol's iodine could also be employed. A vessel 168 provides fluidic connection between the imager reservoir 140 and the diagnostic applicator 125, and a pump mechanism 170 is for forcing the imaging agent through the vessel to the diagnostic applicator for depositing the imaging agent 166 on the examination area 200. The pump mechanism may include a compression trigger and a pressure vessel 172 similar to a spray bottle for simply forcing a spray of acetic acid onto the examination area 200. The compression trigger forces contraction of the elastic pressure vessel 172 for inducing a pressure in the vessel 168 to lightly spray or deposit the imaging agent onto the examination area 200.

Following application of the imaging reagent, a coloration change alerts a skilled practitioner, technician or medical personnel as to positive test denoting a premalignant or malignant lesion, as shown in FIG. 2. The imaging apparatus 130 provides a more detailed image from a line of sight 105B to the display 132, in addition to the line of sight 105A through the examination portal 110 is. As an alternative to visual inspection, the operator may invoke a computerized analysis of the captured image in realtime using the predefined machine learning diagnostic algorithm housed on the device 134. Additionally, the image could also be transmitted wirelessly to a database interface 136-1 (or wired port 136, FIG. 1) to access an image library 138, such that the interface is adapted to receive the captured image for comparison to a repository of preexisting images and a determination of disease from a correspondence to the preexisting images. Various image processing comparison techniques could be compared to determine the presence of premalignant or malignant lesions, which may help in borderline cases where the patches/stripes 212 of coloration and shading are subtle. Additionally, certain configurations may employ a machine learning processor configured for comparing the preexisting images to the captured image for computing a correspondence with the captured image and determining whether the captured image results from a malignancy.

Following a diagnosis of disease, treatment may be readily applied by ablation or excision using electrocautery or similar treatment. The treatment probe 129 further connects to a power supply 180, a switching circuit 182, and the tungsten filament 164 or similar heating element. The heating element connect to the power supply and is operable for activation by the switching circuit for electrocauterization of the examination area. For example, two AA batteries to provide a 3.0-3.2 volt signal can activate a small heating element. The treatment probe therefore employs two electrodes and the tungsten filament 164 for ablation; alternatively, the treatment probe is configured for cryogenic treatment of the examination area 200.

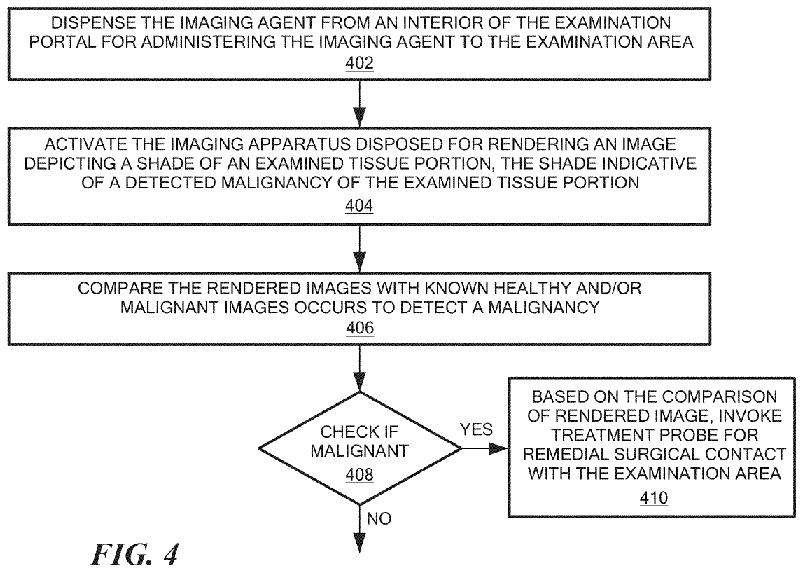

FIG. 4 is a flowchart of usage, diagnosis and treatment using the device of FIGS. 1-3B. Referring to FIGS. 1-4, the method for single transaction diagnosis and treatment of a cervical examination area 200 includes disposing an examination portal defined by an elongated body adapted for visual observation towards the examination area, in which the elongated body contains a comingled treatment probe bundle 148, including the diagnostic applicator 125, the imaging sensor 127, and the treatment probe 129 adapted for therapeutic contact with the examination area 200. At step 402, the diagnostic applicator 125 dispenses the imaging agent 166 from an interior of the examination portal for administering the imaging agent to the examination area 200. This applies the diagnostic reagent from an interior of the device already disposed and focused on the treatment area. The device 100 then activates the imaging apparatus 130 disposed for rendering an image depicting a shade of an examined tissue portion, such that the shade or captured pattern is indicative of a detected malignancy of the examined tissue portion, as depicted at step 404. The examined tissue portion exhibits a color change reaction that may be detected by visual inspection by the examiner. A comparison of the rendered image with known healthy and/or malignant images occurs to detect a malignancy, as shown at step 406. As indicated above, the image rendered may also be subjected to computational analysis by a machine learning diagnostic algorithm built based on comparison of numerous images of normal and precancerous and malignant lesions. A check is performed, at step 408, and based on the comparison of the rendered image indicating a premalignant or malignant lesion, the treatment probe 129 is invoked for remedial surgical contact with the diagnosed lesion in the examination area 200, as depicted at step 410. In contrast to conventional cervical examination and results, rendering of the captured images and activation of the treatment probe occurs during a single examination based on disposing the elongated body adjacent the examination area for access via the examination portal 110, mitigating patient tracking and follow-up.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A cervical examination and treatment device, comprising:
an examination portal defined by an elongated body adapted for visual observation of an examination area;
an imaging sensor disposed in an interior of the examination portal for capturing images of the examination area, the imaging sensor coupled to an imaging apparatus;
a diagnostic applicator disposed in an interior of the examination portal for administering an imaging agent to the examination area; and
a treatment probe adapted for access to the examination area, the treatment probe responsive to an operator control for remedial surgical contact with the examination area,
the treatment probe operable from the operator position concurrently with rendering of the captured images.

2. The device of claim 1 wherein the examination portal further comprises an elongated tube having a distal end for placement adjacent the examination area and a proximate end defining an operator position;
the diagnostic applicator, imaging sensor, and treatment probe concurrently disposed at a distal end of the examination portal.

3. The device of claim 2 wherein the diagnostic applicator, imaging sensor and treatment probe form a bundle coupled to a control member, the control member extending through the elongated body to the proximate end and responsive to operator control of the rigid bundle.

4. The device of claim 3 wherein the elongated body has a pivotal attachment to the control member, the control member further comprising a handle at the proximate end of the elongated body, the distal end responsive to manipulation from the handle via the pivotal attachment.

5. The device of claim 2 further comprising a comingled treatment probe, including
the diagnostic applicator having an imaging nozzle at a distal tip adapted to dispense an imaging substance onto the examination area;
the imaging sensor configured for visual recordation of the examination area, the imaging sensor more proximate than the tip of the imaging nozzle; and
the treatment probe for therapeutic contact with the examination area, the treatment probe more distal than the visual fiber or the imaging nozzle.

6. The device of claim 1 further comprising one or more wings hingedly attached to the distal end of the elongated tube for temporarily displacing soft tissue around the examination area for providing unobstructed visualization and access to the examination area.

7. The device of claim 1 wherein the imaging sensor couples to the imaging apparatus via a signal cable running through an interior of the elongated tube or wirelessly, the imaging apparatus disposed for rendering an image depicting a shade of an examined tissue portion, the indicative of a detected lesion of the examined tissue portion.

8. The device of claim 1 further comprising:
an imager reagent reservoir for storing the imaging reagent;
a vessel for fluidic connection between the imager reservoir and the diagnostic applicator; and
a pump mechanism for forcing the imaging agent through the vessel to the diagnostic applicator for depositing the imaging agent on the examination area.

9. The device of claim 8 wherein the pump mechanism includes a compression trigger and an elastic pressure vessel, the compression trigger forcing contraction of the elastic pressure vessel for inducing a pressure in the vessel.

10. The device of claim 1 wherein the treatment probe further comprises:
a power supply;
a switching circuit; and
a heating element, the heating element connected to the power supply and operable for activation by the switching circuit, the heating element configured for electrocauterization of the examination area.

11. The device of claim 1 wherein the treatment probe is configured for cryogenic treatment of the examination area.

12. The device of claim 1 wherein rendering of the captured images and activation of the treatment probe occur during a single examination based on disposing the elongated body adjacent the examination area for access via the examination portal.

13. The device of claim 1 wherein the remedial surgical contact includes at least one of electrocautery, cryotherapy, laser therapy, knife or surgical ablation or chemical ablation.

14. The device of claim 1 wherein the imaging apparatus further comprises an interface to an image library, the interface adapted to receive the captured image for comparison to a repository of preexisting images and a determination of disease status from a correspondence to the preexisting images.

15. The device of claim 14 further comprising a machine learning processor, the machine learning processor configured for comparing the preexisting images to the captured image for computing a correspondence with the captured image and determining whether the captured image results from a malignancy.

16. The device of claim 15 further comprising an interface to a remote data repository, the interface configured to invoke the interface for computing the correspondence with the captured image.

17. A method for single transaction diagnosis and treatment of a cervical examination area, comprising:
disposing an examination portal defined by an elongated body adapted for visual observation towards an examination area, the elongated body containing a comingled treatment probe, including:
a diagnostic applicator having an imaging nozzle at a distal tip adapted to dispense an imaging substance onto the examination area;
an imaging sensor configured for visual recordation of the examination area, the imaging sensor more proximate than the tip of the imaging nozzle; and
a treatment probe adapted for therapeutic contact with the examination area, the treatment probe more distal than the visual fiber or the imaging nozzle;
dispensing an imaging agent from the diagnostic applicator disposed in an interior of the examination portal for administering the imaging agent to an examination area;
activating an imaging apparatus disposed for rendering an image depicting a shade of an examined tissue portion, the shade indicative of a detected premalignant or malignant lesion on the examined tissue portion; and,
based on the rendered image, invoking the treatment probe for remedial surgical contact with the examination area.

18. The method of claim 17 further comprising dispensing an imaging agent including acetic acid, and based on the rendered image indicating a whitening of the examination area, activating an electrocautery element in the treatment probe for ablating a region indicative of the detected malignancy.

19. A system for standalone, single transaction diagnosis and treatment of a cervical examination area, comprising:
disposing an examination portal defined by an elongated body adapted for visual observation towards an examination area, the elongated body containing a comingled treatment probe bundle, the probe bundle having:
a diagnostic reagent applicator having an imaging nozzle at a distal tip adapted to dispense an imaging substance onto the examination area;
an imaging sensor configured for visual recordation of the examination area, the imaging sensor more proximate than the tip of the imaging nozzle; and
a treatment probe adapted for therapeutic contact with the examination area, the treatment probe more distal than the visual fiber or the imaging nozzle;
an imager reservoir for dispensing an imaging agent from the diagnostic applicator disposed in an interior of the examination portal for administering the imaging agent to an examination area;
an imaging apparatus disposed for rendering an image depicting a shade of an examined tissue portion, the shade indicative of a detected malignancy of the examined tissue portion; and
the treatment probe for invoking remedial surgical contact with the examination area based on the rendered image.
